(19) United States
(12) Patent Application Publication
(10) Pub. No.: US 2008/0187549 A1
(43) Pub. Date: Aug. 7, 2008

(54) Polyantigenic-based (multiple antigens) modi or set of methods for developing infertility vaccines

(76) Inventor: William A. Morey, Pensacola, FL (US)

Correspondence Address: William A. Morey, 3847-A Belle Meade Ct., Pensacola, FL, 32503, US

(21) Appl. No.: 11/703,270

(22) Filed: Feb. 7, 2007

(51) Int. Cl. A61K 39/00

(52) U.S. Cl. 424/184.1

(57) ABSTRACT

Said invention is a unique modi or set of immunological techniques/methods, by which to develop various types of infertility vaccines, primarily designed to reduce fertility or to produce infertility in animals and/or in humans. Said reduced fertility or infertility may be permanent or exists with variable duration. The immunological infertility responses, resulting from said uniquely developed vaccines (as affected/created by the proposed methodologies), may be superior to currently existing infertility vaccines {referenced, herein}, since they (the vaccines resulting from the proposed methods, herein) would contain a far greater diversity/variety of antigens; and are therefore, considered as polyantigenic vaccines, herein. A larger diversity/variety of antigens may better elicit a greater diversity/variety of endogenous antibodies. A greater variety of antibody types and antibody variable sites may enhance the infertility response.

Correspondingly, the proposed methods may result in infertility vaccines which affect greater numbers and types of memory cells. While currently existing infertility vaccines use individual or limited numbers of identified antigens to sperm, or to sex-related hormones, etc. Said proposed modi or set of methods are unique, not only because they are polyantigenic, but because of some of the specified antigenic sources employed, besides sperm, i.e., ova, gamete germinal cells, gamete precursor cells, male ejaculate, liquor folliculi, certain differentiated stem cells, etc. The proposed modi does not use isolated/identified sperm antigens, as is currently used in infertility vaccines. Heretofore, the proposed modi has not been proposed.

Because humans and many domesticated animals (cats, dogs, cattle, sheep, birds, etc.) are such out-bred species, in that they may possess a broad variety of gamete-based antigens, individual responsiveness to an immunologic vaccine which uses a single or highly limited number (one or two) of isolated/identified antigens, is apt to be ineffective. Limited antigen types may fail to adequately address the vaccine recipient's own gamete-based antigens. The use of the proposed polyantigenic modi may enhance vaccine efficacy by providing a greater diversity of antigens, and thereby affecting a broader spectrum of response antibodies, etc.

### 1. FIELD OF THE INVENTION

Issues within this patent application involve disciplines within the fields of biology and/or medical physiology, e.g., immunology, vaccines, birth control, fertility, sex physiology, etc. This document petitions that a utility patent be granted for a modi or set of methodologies (unique biomedical methods/procedures/techniques), designed to create infertility vaccines, to produce either reduced fertility or infertility in animals and/or in humans. Whenever reference is made to infertility vaccine(s) in this patent application, said reference to infertility vaccine(s) may be considered to include both the concept of infertility (temporary and permanent), as well as the concept of reduced fertility (temporary and permanent). The proposed immuno-methodologies employ unique polyantigenic-containing (containing many antigens) infertility vaccine components. The antigen-containing sources, proposed herein, are harvested, processed, and prepared for use in infertility vaccines, by using techniques which already exists or by novel techniques. Said techniques are not a part of this application.

The antigenic vaccine-components, proposed in this application, are not isolated antigens, nor are they identified antigens (not isolated/identified sperm antigens), as are the antigens which have been used in many of the existing infertility vaccines. Isolated and/or identified antigens, to sperm or to the zona pellucida or to the various sex hormones or sex hormone receptors, are not used in this application. The vaccines resulting from the proposed methods are not made by simply using one or a few isolated/identified antigens, but rather by using many/multiple antigens, derived from gametes (sperm and/or ova), and/or gamete support tissues (male ejaculate, and/or liquor folliculi, and/or mucin), and/or precursor cells to gametes, and/or other sex-related tissues and cell types, as derived from differentiated stem cells. These “polyantigenic methods” have never before been utilized to create infertility vaccines.

The infertility vaccines, which may be derived from the proposed methods, are not designed to produce or in anyway promote miscarriage or abortion, nor are they designed to disrupt the development of a fertilized ovum or zygote or embryo, nor to disturb normal endocrine function, as many of the existing (referenced below) vaccines appear to do. But rather, the proposed methods are designed to create infertility vaccines which act to prevent fertilization.

Antisperm antibodies can be made by men and women, and can result in infertility in both sexes (Pertinent Scientific References include: Bhande, S. & Naz, R. K. (2006), Shelton J, Goldberg E., (1985), Shohat M, Hardy B, Mannheimer S, Fisch B, Shohat B. (1996)). These antisperm antibodies may be made when sperm enter the blood or lymph systems by: infection, surgery, injury, etc. While this author found no conclusive reference to antibodies to ovum-based antigens, it is possible that such “antiovum” antibodies exist, since autoimmune oophoritis inhibited fertility in rats which were isoimmunized with homogenates of ovary (Pertinent Scientific Reference: Damjanovic M, Jankovic B D., (1989)). So, it may be reasonable that antibodies could be elicted by ovum-based antigens in a vaccine. Systemic administration of an ovarian homogenate (Pertinent Scientific Reference: Damjanovic M, Jankovic B D., (1989)) might also result in antibodies which could compromise ovarian hormone function, including ovarian hormone production; whereas, antibodies to ovum-based antigens are unlikely to do so.

Efforts have been made to delineate specific sperm antigens which elicit antisperm antibodies (Pertinent Scientific Reference: Bhande, S. & Naz, R. K. (2006)). It is the contention of this proposal that delineation of specific sperm antigens may not be necessary to produce infertility vaccines. Isolated and identified sperm-antigen-based vaccines may well be ineffective since they employ only one or two sperm antigens and therefore are likely to only produce one or two types of response-antibodies, a diversity which may be inadequate at killing gametes. The Proposed infertility vaccine methodologies do not require that specific antigens be isolated or identified, or that synthetic antigens be developed.

### 2. DESCRIPTION OF THE PRIOR ART

The patents and references, sited herein, may not be necessary, since the vaccines, which they reference, are not similar to what is proposed, herein. Many Patents and scientific articles exist concerning infertility vaccines; more than listed here. None have been derived by methods which are similar to what is proposed, herein: nor are any of the existing infertility vaccines similar to the type of vaccine which would be derived by the immune methods which are Proposed in this application. U.S. Patents and Patent Applications are sited for types of infertility vaccines which might be considered distantly related in function to what is proposed, herein; but which in fact, are completely different in the methods of development and vaccine content. The proposed methods for developing infertility vaccines utilize a polyantigenic approach, whereas current immunocontraceptive vaccines employ extremely limited types of antigens: a sperm-based antigen, or a single synthetic peptide as the antigen, or a hormone as the antigen, or a type of hormone receptor as the antigen, etc.

As mentioned, current immunocontraceptive vaccines employ extremely limited types of antigens, i.e., one or two sperm-based antigens (Pertinent U.S. Patents include: Hao, et al (2006) U.S. Pat. No. 7,094,547, Hao, et al (2001) U.S. Pat. No. 6,924,121, Herr, et al (2001) U.S. Pat. No. 6,258,364, Herr, et al (1998) U.S. Pat. No. 5,830,472, Herr, et al (1997) U.S. Pat. No. 5,602,005. Pertinent Scientific Journal References include: Aitken R J, Paterson M, Koothan P T. (1993), Aitken R J. (2002), Bandivdekar A H, Koide S S, Sheth A R. (1991), Bandivdekar A H, Vernekar V J, Mruk D, Cheng C Y, Koide S S, Moodbidri S B. (2001), Chen D, Huang Y. (2004), Coddington C C, Alexander N J, Fulgham D, Mahony M, Johnson D, Hodgen G D., (1992), Diekman A B, Norton E J, Klotz K L, Westbrook V A, Herr J C. (1999), Ge Y F, Huang Y F., (2003), Goldberg E., (1983), Hardy C M, Clydesdale G, Mobbs K J, Pekin J, Lloyd M L, Sweet C, Shellam G R, Lawson M A., (2004), Isahakia M A, Bambra C S., (1992), Jones W R., (1994), Kerr L E., (1995), Lea I A, van Lierop M J, Widgren E E, Grootenhuis A, Wen Y, van Duin M, O'Rand M G., (1998), Li T S., (1974), McCauley T C, Kurth B E, Norton E J, Klotz K L, Westbrook V A, Rao A J, Herr J C, Diekman A B., (2002), McLaughlin EA, Holland M K, Aitken R J., (2003), Naz R K., (1999), Naz R K., (2000, 2002, 2004, 2005, 2006), Naz R K, Bhargava K K., (1990), Naz R K, Chauhan S C., (2001), Naz R, Menge A., (1990), Naz R K, Zhu X, Kadam A L., (2000), O'Rand M G, Beavers J, Widgren E E, Tung K S., (1993), Pavlou S N., (1994), Primakoff P, Lathrop W, Woolman L, Cowan A, Myles D., (1988), Primakoff P, Woolman-Gamer L, Tung K S, Myles D G., (1997), Suri A., (2004), Talwar G P., (1978), Talwar G P, Naz R K., (1981)).

Some studies (Bandivdekar A H, Vernekar V J, Kamada M, Raghavan V P., (2005), Carelli C, Audibert F, Gaillard J, Chedid L., (1982), Jones W R., 1994), Naz R K., (2004), O'Rand M G, Beavers J, Widgren E E, Tung K S., (1993)) have used, or proposed using, a synthetic peptide antigen. Other vaccines have been made by using an isolated antigen from the zona pellucida, a ZP antigen (Pertinent U.S. Patents include: Chi, et al (2006) U.S. Pat. No. 7,148,021, Dong, et al (2002 and 2001) U.S. Patent Application #s 20020172982, 20020028470, Dunbar, et al (2001) U.S. Pat. No. 6,264,953, Dunbar, et al (1997) U.S. Pat. No. 5,637,300, Harris, et al (1999) U.S. Pat. Nos. 6,027,727 and 6,001,599 and 5,989,550 and 5,981,228 and 5,976,545. Pertinent Scientific References include: Aitken R J, Paterson M, van Duin M., (1996), Bagavant H, Fusi F M, Baisch J, Kurth B, David C S, Tung K S., (1997), Caudle M R, Shivers C A., (1989), Cui X, Duckworth J., (2005), Hardy C M, ten Have J F, Mobbs K J, Hinds L A., (2002), Hasegawa A, Koyama K, Inoue M, Takemura T, Isojima S., (1992), Kaul R, Afzalpurkar A, Gupta S K., (1996), Kerr P J, Jackson R J, Robinson A J, Swan J, Silvers L, French N, Clarke H, Hall D F, Holland M K., (1999), Kitchener A L, Edds L M, Molinia F C, Kay D J., (2002), Lou Y, Ang J, Thai H, McElveen F, Tung K S., (1995), Lou Y H, Bagavant H, Ang J, McElveen M F, Thai H, Tung K S., (1996), Mahi-Brown C A., (1996), Miller L A, Johns B E, Killian G J., (1999), Miller L A, Killian G J., (2002), Naz R K, Ahmad K., (1994), Paterson M, Jennings Z A, Wilson M R, Aitken R J., (2002), Paterson M, Wilson M R, van Duin M, Aitken R J., (2002), Paterson M, Wilson M R, van Duin M, Aitken R J., (1996), Paterson M, Wilson M R, Jennings Z A, van Duin M, Aitken R J., (1999), Paterson M, Wilson M R, Morris K D, van Duin M, Aitken R J., (1998), Ringleb J, Rohleder M, Jewgenow K., (2004), Shigeta M, Hasegawa A, Hamada Y, Koyama K., (2000), Tesarik J., (1995), Tung K S, Ang J, Lou Y., (1996), Turner J W Jr, Liu I K, Flanagan D R, Bynum K S, Rutberg A T., (2002), No Author Listed, (1991), the last reference listed in the reference section, herein).

A few studies have investigated the use of the epididymis to provide an antigen source, in the creation of a male contraceptive (Pertinent Scientific References include: Cui Y G, Tong J S, Wang X H (2002), Khole V., (2003)). Such an epididymis-based vaccine may result in undesirable damage to the epididymis. In addition, a temporarily-acting antifertility effect has been attempted using the passive administration of antibodies (Pertinent Scientific Reference: Bandivdekar A H, Vernekar V J, Kamada M, Raghavan V P., (2005)).

Some infertility vaccines have been made, using a fertility-related hormone as an antigen source, i.e., chorionic gondadotropin (CG) (Pertinent Scientific References include: Chen Y, Liu Z, Yang Y, Chen Y Z, Peng J P., (2002), Hulme M J, Fehilly C B, Hearn J P., (1980)), and gonadotrophic releasing hormone (GnRH) and gonadotrophic hormones (luteinizing hormone {LH} and follicle stimulating hormone {FSH}) (Pertinent Scientific References include: Awoniyi C A., (1994), Ferro V A, O'Grady J E, Notman J, Stimson W H., (1995), Ladd A, Tsong Y Y, Walfield A M, Thau R., (1994), Ladd A, Walfield A, Tsong Y Y, Thau R., (1995), Moudgal N R, Murthy G S, Prasanna Kumar K M, Martin F, Suresh R, Medhamurthy R, Patil S, Sehgal S, Saxena B N., (1997), Moudgal N R, Ravindranath N, Murthy G S, Dighe R R, Aravindan G R, Martin F., (1992), Sairam M R, Krishnamurthy H., (2001), Saxena B B, Clavio A, Singh M, Rathnam P, Bukharovich Y, Reimers T Jr, Saxena A, Perkins S., (2002), Tung K S, Teuscher C., (1995)). One vaccine component was made using antigens from placental tissue (Pertinent Scientific Reference: Hardy C M, Clydesdale G, Mobbs K J., (2004)). The above mentioned placenta-based vaccine, and the vaccines made in response to antigens of the zona pellucida (above) or to human chorionic gondadotropin or to FSH or to LH (above) may not act to prevent fertilization, but may function by eliciting the formation of endogenous antibodies which may act to destroy the developing zygote or fertilized egg, and therefore such antigens-based vaccines may incur some degree of social controversy.

Moreover, the infertility vaccines which function by altering gonadgotrophic releasing hormones, LH or FSH, or by altering said hormone receptors (Pertinent Scientific Reference: Saxena B B, Clavio A, Singh M, Rathnam P, Bukharovich Y, Reimers T Jr, Saxena A, Perkins S., (2002)) are likely to influence much more than fertility, since critical sex steroid hormones (estrogen, progesterone, and testosterone) are directly dependant on these hormones for their endogenous production.

### 3. SUMMARY OF THE INVENTION

Said invention is a modi or set of immunological techniques, by which to create various types of infertility vaccines, designed to reduce animal and/or human fertility or to produce infertility in animals and/or in humans, permanently or with varying duration. The determination of the degree of infertility or duration of infertility of said resultant vaccines is not a part of this application. The immunological infertility response, resulting from said infertility vaccines, as created by the proposed methodologies, may be superior to existing infertility vaccines {referenced above}, since they (the vaccines resulting from the proposed methods) would be designed to contain a greater diversity/variety of antigens, than the infertility vaccines (above-referenced) created, thus far. A larger diversity/variety of antigens may better insure a greater diversity/variety of endogenously elicited response-antibodies (antibodies with different variable sites, etc.). Correspondingly, greater numbers and types of memory cells may be created in response to the infertility vaccines, resulting from the proposed methods.

Conversely, the fewer the types of antigens contained within an infertility vaccine, the fewer the types of endogenously-made response antibodies and the less effective such a vaccine is apt to be. The use of limited types of antigens may account for the lack of success and/or the reduced efficacy of the existing infertility vaccines.

The proposed set of immune methodologies, for creating infertility vaccines, utilizes various combinations and concentrations of the following potential antigen-containing sources/factors (below). Said antigens sources may be animal and/or human, and may be live and/or dead, and may be autologous, and/or allogenic, and/or trans-specie (derived from specie(s) other than the specie of the vaccine recipient), may be derived from differentiated stems cells, provided there are no legal restrains, and may be any combinations and/or concentration of said antigen sources.

Proposed Antigen Sources

1. Gametes
Male, and/or female (sperm and/or ova)
Live, and/or dead gametes
Autologous, and/or allogenic, and/or trans-specie
Whole, and/or fractioned/fractionated, and/or digested, and/or chemically separated, and/or concentrated

2. Gamete germinal cells (GGCs) and/or gamete-precursor cells (GPCs)
Male, and/or female
Live, and/or dead
Autologous, and/or allogenic, and/or trans-specie
Whole, and/or fractioned/fractionated, and/or digested, and/or chemically separated, and/or concentrated

3. Male ejaculate fluid (MEF)
Containing or not containing the sperm
Autologous, and/or allogenic, and/or trans-specie
Whole, and/or fractioned/fractionated, and/or digested, and/or chemically separated, and/or concentrated

4., Female liquor folliculi (FLF)
Containing or not containing ova
Autologous, and/or allogenic, and/or trans-specie
Whole, and/or fractioned/fractionated, and/or digested, and/or chemically separated, and/or concentrated

5. Female mucin {a mucoploysaccharide} (MU)
Autologous, and/or allogenic, and/or trans-specie
Whole, and/or fractioned/fractionated, and/or digested, and/or chemically separated and/or concentrated

6. Differentiated stem cells (DSCs)
Male and/or female
Autologous, and/or allogenic, and/or trans-specie
Whole, and/or fractioned/fractionated, and/or digested, and/or chemically separated and/or concentrated
Differentiated into: sperm, and/or ova, and/or germinal precursor cells, and/or immature gametes.
Fractioning, or apportioning, or separating, or concentrating said cells, or tissues, or fractions or portions thereof, may be accomplished by technical means which are not part of this patent; e.g., ultrasound fractionation, chemical or enzymatic separation or digestion, chromatography, centrifugation, iontophoresis, etc. Said techniques may later be useful in determining and creating the efficacy of the resultant vaccines. The exact combinations and concentrations of the above antigen-containing factors/sources would need to be determined by research.

The proposed methods of this application employ the aforementioned antigen sources which may be used to develop infertility vaccines. The importance of said antigen sources and their interrelationships are exemplified and explained in more detail in disclosure 5. (Detailed Description of the Preferred Embodiment); the scope of which will be indicated in the appended claims, to follow.

The proposed modi or methodologies are designed to produce infertility vaccines which function by producing an endogenous immune response, against antigens derived from gametes (sperm and/or ovum) and/or from gamete creating or gamete supporting factors/cells/tissues (ejaculate, mucin, liquor folliculi, gamete germinal cells, immature gametes, etc.) and/or from antigens derived from certain differentiated stem cells (provided legal restrictions do not exist). The selection of these (above-described) antigen sources may product an immuno-infertility response, prior to fertilization. Consequently, (as previously stated) the proposed methodologies are not designed to produce an infertility vaccine which would abort a zygote, or an embryo, but which would destroy a gamete, prior to fertilization. Moreover, the proposed methods are specifically designed to create vaccines which have no significant influence on endogenous hormone production. In these two respects (non-abortion effect and non-hormonal effect), said proposed methodologies and the resultant infertility vaccines may be less apt to incur social controversy.

Possible Uses or Benefits of Vaccines Derived from Proposed Methods/Techniques

An effective immunologic infertility vaccine may have multiple uses or benefits and possibly more than listed below:
1. For persons with children, who do not wish additional children, and do not want to undergo sterilization surgery or to use birth control methods (pharmaceutical, mechanical devices, etc.)
2. For older persons, who do not desire to bare or father children, and do not wish to undergo infertility surgery or use birth control (pharmaceutical, mechanical devices, etc.)
3. Reduction of certain types of age-related birth defects or physiological abnormalities
   Older humans and animals (males and females) are more likely to produce offspring with certain types of physiological abnormalities; hence, a viable infertility vaccine would have the effect of reducing the population levels of said abnormal offspring. Age-related abnormalities in the offspring may become of greater concern with the recent development of more effective pharmaceutical treatments for erectile dysfunction, and the resultant increase in the frequency of sex in older fertile males.
4. To control wild animal populations; most importantly, where the environmental interspecies balance has been grossly disturbed and is adversely impacting the environment or society
5. To control feral animal populations
6. To control populations of domesticated animals or pets

Potential Vaccine Candidates

Potential vaccine candidates may be organisms (human and animal) capable of forming b-cell antibodies and/or other immunological responses (e.g., neutrophils, t-cells, monocytes, etc): to the proposed antigen sources.

Trans-Specie Antigen Sources

Trans-specie antigens may be effective in eliciting infertility, in human and in other animals, since the gametes or other sex-related tissues (described below), may share identical or highly similar antigenic molecules, e.g., proteins, complex carbohydrates, etc.

### 4. BRIEF DESCRIPTION OF THE DRAWINGS

No drawings are used to describe the proposed utility application/invention.

### 5. DETAILED DESCRIPTION OF THE PREFERRED EMBODIMENT

Antigen-containing biological factors (cells, tissues, etc), used in the proposed methods for developing infertility vaccines include:

1. Gametes
Male, and/or female (sperm and/or ova)
Live, and/or dead gametes
Autologous, and/or allogenic, and/or trans-specie
Whole, and/or fractioned/fractionated, and/or digested, and/or chemically separated, and/or concentrated

2. Gamete germinal cells (GGCs) and/or gamete-precursor cells (GPCs)
Male, and/or female
Live, and/or dead
Autologous, and/or allogenic, and/or trans-specie
Whole, and/or fractioned/fractionated, and/or digested, and/or chemically separated, and/or concentrated

3. Male ejaculate fluid (MEF)
Containing or not containing the sperm
Autologous, and/or allogenic, and/or trans-specie
Whole, and/or fractioned/fractionated, and/or digested, and/or chemically separated, and/or concentrated

4., Female liquor folliculi (FLF)
Containing or not containing ova
Autologous, and/or allogenic, and/or trans-specie
Whole, and/or fractioned/fractionated, and/or digested, and/or chemically separated, and/or concentrated

5. Female mucin {a mucoploysaccharide} (MU)
Autologous, and/or allogenic, and/or trans-specie
Whole, and/or fractioned/fractionated, and/or digested, and/or chemically separated and/or concentrated

6. Differentiated stem cells (DSCs)
Male and/or female
Autologous, and/or allogenic, and/or trans-specie
Whole, and/or fractioned/fractionated, and/or digested, and/or chemically separated and/or concentrated
Differentiated into: sperm, and/or ova, and/or germinal precursor cells, and/or immature gametes.

Antigen sources, as listed and described above [0005], may be utilized in all possible, amounts, concentrations, and/or combinations. The exact amounts, concentrations, and combinations of said antigen sources may be established by research; said research is not a part of this application.

The listed antigenic sources [0005] employed in the proposed methods may come from a single specie (human and/or animal) or from various species. Said antigenic sources may come from the same specie, as the specie receiving the vaccine, or may contain antigens derived from one or more other species. The resultant vaccines may contain specie-specific antigens or trans-specie antigens or any combination, thereof. Using species, other than the specie receiving the proposed vaccine, may prove to be immunologically efficacious, as well as cost effective. For example, antigens (proteins, complex carbohydrates, etc.), from the gametes of one specie, may also exist on the gametes of a different specie. Therefore, the gamete-based antigens of one species may be immunologically responsive in another. For example, the gametes (sperm and/or ova) of the sperm whale (a mammal) may possess antigens capable of eliciting a significant infertility-producing antibody response in humans (human males and/or human females). Such an infertility vaccine, might be named the “Moby Dick Vaccine.”

Gamete germinal cells (GGCs) and/or gamete-precursor cells (GPCs) are defined herein as follows: GGCs are testicular and/or follicular ovarian cells which create the first step of meiosis for spermatogenesis or oogenesis, when they (GGCs) divide. GPCs are the cells derived from said germinal cells, having undergone partial meiosis, but are not yet mature gametes; said GPCs may include polar bodies, in the case of oogenesis. Of all the antigen sources mentioned in this proposal, GGCs may produce the undesirable side effect of altering hormone sex steroid hormone production, and may therefore be excluded for initial consideration for use in the research for an experimental infertility vaccine.

GGCs and/or GPCs, and/or fractions/portions thereof, may be used individually or together with the other antigen sources described herein, as part of various infertility vaccines. Moreover, GGCs could be used to culture GPCs, as well as to culture gametes, to then be used as antigen sources in the proposed methods.

Male ejaculate fluid (MEF), containing or not containing sperm and/or fractions/portions thereof, and/or the female liquor folliculi (FLF, containing or not containing ova) and/or fractions/portions thereof, and/or female mucin (MU), a mucoploysaccharide and/or fractions/portions thereof, may be used individually or in all the various possible combinations, as all or as part of the methodology for developing infertility vaccines. Moreover, these described antigen sources [0010] they be used in various combinations with the other antigen sources described, herein [0005]. Said MEF, FLF, and MU may serve as antigen sources for producing infertility vaccines; since these antigen sources act to support gamete function, to immunologically destroy said tissues may compromise the survival of the associated gametes.

The use of differentiated stem cells (DSCs) may be contingent upon legalization. Stem cells which could be differentiated into male or female gametes, and/or into male and/or female GGCs, and/or into male or female GPCs, and may be employed as antigen sources in the proposed methods, as described herein {[0005] through [0010]}. Said DSCs may be used whole and/or in various fractions and in different concentrations and combinations, as well as in various combinations and concentrations with the other aforementioned antigen sources of this proposal.

The above-mentioned [0005] antigen sources may be derived by various scientific extraction or procurement or harvesting techniques, which are not a part of this application. The above-mentioned [0005] antigen sources may be derived from whole and/or partial tissues, and/or whole or partial cellular sources. Said antigen sources may be fractioned/fractionated, and/or chemically separated, and/or chemically or enzymatically digested into varying amounts/concentrations, and/or into various combinations and concentrations for use infertility vaccines. Said fractions or portions or concentrations of the aforementioned antigen sources may be made by using various existing or novel means/techniques (ultrasound, chemical, electric current, light, heat, cold, digestive enzymes, solvents, ionophoresis, centrifugation, chelation, dilution, etc.), so as to develop vaccines with the desired level of immuno-responsiveness. For example, it may be determined that more efficacious (endogenous antibody-producing) antigens are contained on and/or in the head of the sperm, or on the midsection of the sperm, or in a particular fraction of a digested or chemically separated portion of the sperm, or on the plasma membranes of the ova, or in the protein portions of the male ejaculate, or on the heavier fractions of the enzymatically or chemically digested plasma membranes of the female GGCs, or the lighter protein-containing extracted solutions of the immature gametes, or a certain protein-containing extract of mucin, or in various combinations of antigen source fractions which contain molecules which are apt to be antigenic, etc.

Certain portions or factions or concentrations or combinations of the various antigen sources [0005] may be found to induce infertility but with variable durations of effect. Such preferable fractions or portions may be separated and collected/managed by various techniques, and said techniques are not part of this application, and are in no way an attempt to isolate a particular antigen.

Related factors which are not included in this patent application, include:
A. Vaccine delivery/administration vehicles and/or techniques, i.e., intramuscular injection, subcutaneous injection, oral, transdermal absorption, inhalation, intraperitoneal injection, etc., are not a part of this patent application.
B. Sterile or antimicrobial techniques which might be used in the preparation and/or administration of the resultant vaccines are not a part of this application.
C. Antimicrobial agents which may be added to or included within the resultant vaccines are not a part of this application.
D. Resultant vaccines may need to be tested to determine safety, efficacy, and duration of effect. Said testing is not a part of this patent application.
E. The vaccine's non-antigen components, that or those material(s), medium(s), solution(s) which form the matrix in which the antigens are contained, which may include such factors as: preservatives, dissolving or suspensory/suspending material(s), buffering agents, etc., are not part of this patent application.
F. Safety (toxicology, inflammatory aspects, prophylactic pregnancy testing, etc.) and efficacy (degree of infertility and duration of effect) of the proposed methods is not a part of this application.

By utilizing “individualized” autologous antigens, the efficacy of an infertility vaccine may be enhanced. One way that using autologous antigens may better assure infertility is that autologous antigens may increase endogenously-created response antibodies with variable sites which specifically bind said known antigens on the individual's autologous cells/tissues. Such autologous vaccines may offer the vaccine recipient a type of psychological comfort for it may be that certain individuals will not like the concept of gametes from other individuals or animals being introduced into his or her physiology.

A purely autologous human male infertility vaccine would include antigens derived solely from his own (the vaccine recipient's own) cells and tissues, “self-derived:” sperm and/or sperm fractions, and/or sperm germinal cells, and/or immature sperm, and/or fractions of said sperm germinal and/or precursor cells, and/or ejaculate, and/or ejaculate fractions, and/or differentiated (into sperm, sperm germinal cells, and/or immature gametes/gamete precursor cells) stem cells, and/or self-derived differentiated stem cell fractions, and/or any combination and/or concentration of the above mentioned autologous antigen sources.

In accordance with the proposed methods, a male infertility vaccine may be derived from autologous antigens sources, and/or allogenic antigen sources, and/or animal (trans-specie) antigen sources. Consequently, a male infertility vaccine, as developed by the proposed methods, may contain antigens which are: autologous, allogenic, from animal(s) (trans-specie), or any combination of these antigen sources, and in all possible amounts, proportions, fractions/portions, and concentrations.

An autologous infertility vaccine for females may contain various combinations and concentrations and/or fractions of antigens derived from her own: ova and/or ova cell fractions, ovum germinal cells and/or ovum germinal cell fractions, ovum precursor cells (including polar bodies) and/or cell fractions of ovum precursor cells (including polar bodies), and/or differentiated (into ova, ovum precursor cells, and/or ovum germinal cells) stem cells and/or DSC fractions, and/or liquor folliculi and/or liquor folliculi fractions, and/or muscin and/or muscin fractions, in all possible amounts, combinations, and concentrations, thereof.

In accordance with the proposed methodologies, a female infertility vaccine may be derived from autologous antigens sources, and/or allogenic antigen sources, and/or animal (trans-specie) antigen sources. Consequently, a female infertility vaccine, as developed by the proposed methodologies, may contain antigens which are: autologous, allogenic, from animals (trans-specie), or any combination of these three antigen sources, and in all the various amounts, proportions, fractions/portions, and concentrations. Moreover, female infertility vaccines, as derived from the proposed methods, may also contain various combinations and concentrations of female and/or male-based antigens: ova and/or ova fractions, sperm and/or male sperm fractions, and/or male and/or female GGCs, and/or male and/or female GGCs fractions, and/or male and/or female GPCs, and/or male and/or female GPCs fractions, and/or male ejaculate and/or portions of said ejaculate, and/or liquor folliculi and/or portions of said liquor folliculi, to thereby enable the female vaccine recipient to form antibodies or other immune responses: to female gametes (ova), and/or to female GGCs, and/or to female GPCs, and/or to male gametes (sperm), and/or to antigenic factors within the male ejaculate, and/or to antigenic factors within the liquor folliculi.

Said male antigen sources (sperm, sperm fractions, male GGCs, male GGC fractions, male GPCs, male GPC fractions, male ejaculate, and portions of male ejaculate) which may be included in a female infertility vaccine, may be derived from a human male of the vaccine recipient's own choosing. This allowed selection of male antigens may have advantages: 1. The antibody-medicated response is more apt to selectively address the recipient's sex partner, while still affording the recipient a degree of infertility with other potential sex partners. 2. The vaccine recipient may find it to be more psychologically comforting to receive sex-based antigens from the male of her choosing.

Female infertility vaccines, which contain both female and male antigens/antigen sources, may be more likely to produce effective infertility, than those female infertility vaccines which only contain female antigen sources, since the female may produce antibodies to both the invasive sperm antigens, to antigens within the gamete-supporting ejaculate, as well as to her endogenously derived antigenic molecules.

Because of the large number of potentially different antigens [compared to the number of types of antigens used in the preparation of the aforementioned current infertility vaccines (above-referenced)], derived from the proposed antigen sources herein [0005], the resultant vaccines may stimulate a greater (than existing infertility vaccines) endogenous immunologic response, not only in the number and in the diversity/variety of B-cells, and/or diversity/variety of B-cell response-antibodies, but perhaps in the numbers and types of other white blood cells, e.g., T-cells, etc., which may somehow additionally contribute to an infertility response.

The various proposed vaccine antigen sources [0005] may increase the number and types of the corresponding myeloid and/or lymphatic “memory cells” (the precursor cells to vaccine response B-cells {plasma cells} and T-cells, etc.), which may remain alive as an immunological reserve for varying amounts of times, perhaps for a life-time. Depending on the duration of survival of said memory cells, as well as other factors, “booster” administrations of the infertility vaccines, to prolong infertility, may or may not be required. The determination of the duration of survival of said memory cells and the development of the corresponding issue of “booster” administrations are not a part of this proposal.

The proposed immunologic contraception methodologies may not produce the controversial situation of the abortion of a zygote, embryo, or fetus; since, the proposed vaccines are not designed to elicit antibodies or other immune mechanisms which attack molecular factors on or in a zygote, embryo, or developing fetus. Such an “abortion-type” effect may occur with the existing aforementioned (above-referenced) vaccines, derived from antigens of the zona pellucida, the placenta, human chronic gondadotropin (HCG), LH, or FSH, or to antigens derived from the receptors of said hormones. Moreover, since neither hormone producing cells (with the aforementioned exception of GGCs) nor hormone receptors, are used as antigen sources in the proposed methods, the resultant vaccines are much less apt to produce untoward endocrine effects.

What is claimed:

1. This application claims the use of whole live sperm as a possible component in the creation of various infertility vaccines. Said sperm may be human (autologous and/or allogenic), and/or animal (trans-specie; other than human), or any combination of these sperm sources. Said whole live sperm may be used as the only type of antigen source in the infertility vaccine or may be used in various amounts and concentrations, and in various combinations and concentrations with the various other antigen sources, mentioned herein.
This application claims the use of whole dead sperm as a possible component in the creation of various infertility vaccines. Said dead whole sperm may be human (autologous and/or allogenic), and/or animal (trans-specie, other than human), or any combination of these sperm sources. Said whole dead sperm may be used as the only type of antigen source in infertility vaccines or may be used in various amounts and concentrations, and in various combinations and concentrations with the other antigen sources, mentioned herein.
This application claims the use of sperm fractions and/or biochemical components of said sperm, as components in various infertility vaccines. Said sperm fractions may be human (autologous and/or allogenic), and/or animal (trans-specie, other than human), or any combination of these sperm fraction sources. Said sperm fractions may be used as the only type of antigen source in the infertility vaccine or may be used in various amounts and concentrations, and in various combinations and concentrations with the various other antigen sources, mentioned herein.
Methods of fractioning or separating or digesting or concentrating said sperm fractions are not a part of this application. Examples of said fractions may be: the head of the sperm, the tail of the sperm, the partially digested plasma membrane of the sperm, the separated protein fraction(s) of the sperm, the complex carbohydrates fraction of the sperm, etc. This application does not claim the use of a particular isolated sperm-based protein or set of isolated sperm-based proteins.
This application claims the use of male ejaculate (with and/or without sperm) or fractions or portions of said ejaculate, as possible components in various infertility vaccines. Said male ejaculate and/or male ejaculate fractions may be human (autologous and/or allogenic), and/or animal (trans-specie, other than human), or any combination of these sources of male ejaculate.
Said male ejaculate and/or male ejaculate fractions may be used as the only type of antigen source in the infertility vaccine or may be used in various amounts and concentrations, and in various combinations and concentrations with the various other antigen sources, mentioned herein.
This application claims the use of whole live ova as possible components in the creation of various infertility vaccines. Said ova may be human (autologous and/or allogenic), and/or animal (trans-specie, other than human), or may be used in any combination of these ova sources. Said whole live ova may be used as the only type of antigen source in the infertility vaccine or may be used in various amounts and concentrations, and in various combinations and concentrations with the various other antigen sources, mentioned herein.
This application claims the use of whole dead ova as possible components in the creation of various infertility vaccines. Said dead ova may be human (autologous and/or allogenic), and/or animal (trans-specie, other than human), or any combination of these ova sources. Said whole dead ova may be used as the only type of antigen source in the infertility vaccine or may be used in various amounts and concentrations, and in various combinations and concentrations with the various other antigen sources, mentioned herein.
This application claims the use of the fractions and/or biochemical components of ova, as possible components in the creation of various infertility vaccines. Said ova fractions may be human (autologous and/or allogenic), and/or animal (trans-specie, other than human), or any combination of these ova fraction sources. Said ova fractions may be used as the only type of antigen source in the infertility vaccine or may be used in various amounts and concentrations, and in various combinations and concentrations with the various other antigen sources, mentioned herein.
Methods of fractioning or separating or digesting or concentrationing said ova are not a part of this application. Examples of said fractions may be: the partially digested plasma membrane of the ova, the protein fraction(s) of the ova, the complex carbohydrates fraction(s) of the ova, etc. But in no way does this application claim the use of only a particular isolated ovum-based protein or set of isolated ovum-based proteins. Said ova fractions or biochemical components may be derived from human ova (autologous and/or allogenic), and/or from animal (other than human) ova, or from any combination, thereof.
This application claims the use of all or portions/fractions of mucin as possible components in the creation of various infertility vaccines. Said mucin may be human (autologous and/or allogenic), and/or animal (trans-specie, other than human), or any combination of these mucin sources. Said mucin and/or mucin fractions may be used as the only type of antigen source in the infertility vaccine or may be used in various amounts and concentrations, and in various combinations and concentrations with the various other antigen sources, described herein.
This application claims the use of all or portions/fractions of the liquor folliculi (with and/or without ova) as possible components in the creation of various infertility vaccines. Said liquor folliculi may be human (autologous and/or allogenic), and/or animal (trans-specie, other than human), or any combination of these sources of liquor folliculi. Said liquor folliculi may be used as the only type of antigen source in the infertility vaccine or may be used in various amounts and concentrations, and in various combinations and concentrations with the various other antigen sources, described herein.
This application claims the use of whole or portions/fractions of live and/or dead, male and/or female, gamete germinal cells (GGCs) or immature gametes (GPCs), including polar bodies, as possible components in the creation of various infertility vaccines. Said gamete germinal cells or immature gametes may be human (autologous and/or allogenic), and/or animal (trans-specie, other than human), or any combination of these sources of GGCs and GPCs. Said gamete germinal cells or immature gametes, or fractions there of, may be used as the only type of antigen source in the infertility vaccine or may be used in various amounts and concentrations, and in various combinations and concentrations with the other antigen sources, described herein.
This application claims the use of differentiated stem cells and/or fractions/portions thereof as possible components in the creation of various infertility vaccines. Said differentiated stem cells may be derived from human (autologous and/or allogenic), and/or animal (trans-specie, other than human), or any combination of these stem cell sources. Said differentiated stem cells, or fractions there of, may be used as the only type of antigen source in the infertility vaccine or may be used in various amounts and concentrations, and in various combinations and concentrations with the various other antigen sources described, herein. Said differentiated stem cells may have been differentiated into any of the following: sperm, ova, immature gametes (including polar bodies), and/or gamete germinal cells. Said differentiated stem cells may be used as the only antigen source in the infertility vaccine or may be used in various amounts and concentrations, and in various combinations and concentrations with the various other antigen sources, described herein.
This application claims the use of any and all of the aforementioned antigen sources {[0005 through [0034]}, in all of the possible combinations and concentrations, as possible components in the creation of various infertility vaccines, without claiming the use of an identified or isolated sperm antigen.
